(19) United States
(12) Patent Application Publication
(10) Pub. No.: US 2023/0238089 A1
(43) Pub. Date: Jul. 27, 2023

(54) MEDICAL CHECKUP PRESENTATION APPARATUS, MEDICAL CHECKUP PRESENTATION METHOD AND RECORDING MEDIUM

(71) Applicants: NEC Corporation, Minato-ku, Tokyo (JP); NEC Solution Innovators, Ltd., Tokyo (JP)

(72) Inventors: Masahiro HAYASHITANI, Tokyou (JP); Masahiro KUSO, Tokyo (JP); Akihiko SHIBANO, Tokyo (JP); Takayuki BANNO, Tokyo (JP); Junichi YAHARA, Tokyo (JP); Akira YAMAUCHI, Tokyo (JP); Hideyuki TAKETA, Tokyo (JP); Kento SOMA, Tokyo (JP)

(73) Assignees: NEC Corporation, Minato-ku, Tokyo (JP); NEC Solution Innovators, Ltd., Tokyo (JP)

(21) Appl. No.: 18/007,778

(22) PCT Filed: Apr. 22, 2021
(86) PCT No.: PCT/JP2021/016351

§ 371 Date: Dec. 2, 2022

(30) Foreign Application Priority Data
Jun. 4, 2020 (JP) 2020-097582

(51) Int. Cl. G16H 10/60; G16H 50/20

(52) U.S. Cl. CPC G16H 10/60; G16H 50/20

(57) ABSTRACT

A medical checkup presentation apparatus (1) includes: a selection unit (121) for selecting, as a recommended medical checkup, a medical checkup that is recommended to a medical checkup target person based on medical chart data (1110) including an information (1112) related to a history of a medical examination for the medical checkup target person and past medical checkup data (1120) including an information related to a history of a medical checkup; and presentation unit (122) for presenting, to a presentation target person, a recommended medical checkup information including an information related to an item name of the recommended medical checkup.

### TECHNICAL FIELD

This disclosure relates to a technical field of a medical checkup presentation apparatus, a medical checkup presentation method and a recording medium that are configured to select a medical checkup which a medical checkup target person is recommended to take and to present the selected medical checkup to the medical checkup target person.

### BACKGROUND ART

A medical checkup presentation apparatus that is configured to present, to a presentation target (typically, a medical checkup target person), a medical checkup which the medical checkup target person is recommended to take, for the purpose of a prevention or an early detection of a disease of the medical checkup target person is known. For example, the Patent Literature 1 discloses a medical checkup presentation apparatus that is configured to select a medical checkup which the medical checkup target person should take based on a result of a medical interview for the medical checkup target person in the past and a result of the medical checkup for the medical checkup target person in the past, and to display it as a recommended medical checkup on an electronic terminal.

Additionally, there is a Patent Literatures 2 to 5 as a background art document related to this disclosure.

### CITATION LIST

### Patent Literature

Patent Literature 1: JP2009-031973A
Patent Literature 2: JP2017-049985A
Patent Literature 3: WO2008/068914A
Patent Literature 4: JP2008-521130A
Patent Literature 5: JP2001-118014A

### SUMMARY

### Technical Problem

It is desired that the medical checkup presentation apparatus properly select the medical checkup that is recommended to the medical checkup target person. An existing medical checkup presentation apparatus including the medical checkup presentation apparatus disclosed in the Patent Literature 1 has a room for improvement in that the medical checkup recommended to the medical checkup target person is properly selected.

It is an example object of this disclosure to provide a medical checkup presentation apparatus, a medical checkup presentation method and a recording medium that are configured to solve the above described technical problem. As one example, it is an example object of this disclosure to provide a medical checkup presentation apparatus, a medical checkup presentation method and a recording medium that are configured to properly select a medical checkup that is recommended to a medical checkup target person.

### Solution to Problem

One example aspect of a medical checkup presentation apparatus of this disclosure is includes: a selection unit that is configured to select, as a recommended medical checkup, a medical checkup that is recommended to a medical checkup target person based on medical chart data including an information related to a history of a medical examination for the medical checkup target person and past medical checkup data including an information related to a history of a medical checkup; and a presentation unit that is configured to present, to a presentation target person, a recommended medical checkup information including an information related to an item name of the recommended medical checkup.

One example aspect of a medical checkup presentation method of this disclosure is includes: selecting, as a recommended medical checkup, a medical checkup that is recommended to a medical checkup target person based on medical chart data including an information related to a history of a medical examination for the medical checkup target person and past medical checkup data including an information related to a history of a medical checkup; and presenting, to a presentation target person, a recommended medical checkup information including an information related to an item name of the recommended medical checkup.

One example aspect of a recording medium of this disclosure is a recording medium on which a computer program that allows a computer to execute: a selection step at which a medical checkup that is recommended to a medical checkup target person is selected as a recommended medical checkup based on medical chart data including an information related to a history of a medical examination for the medical checkup target person and past medical checkup data including an information related to a history of a medical checkup; and a presentation step at which a recommended medical checkup information including an information related to an item name of the recommended medical checkup is presented to a presentation target person is recorded.

### Effect

The medical checkup presentation apparatus, the medical checkup presentation method and the recording medium described above are capable of properly selecting the medical checkup that is recommended to the medical checkup target person.

### BRIEF DESCRIPTION OF DRAWINGS

FIG. 1 is a block diagram that illustrates an entire configuration of a medical checkup presentation system in a present example embodiment.

FIG. 2 is a block diagram that illustrates a configuration of a medical checkup presentation server in the present example embodiment.

FIG. 3 is a data structure diagram that illustrates one example of a data structure of medical chart data.

FIG. 4 is a data structure diagram that illustrates one example of a data structure of past medical checkup data.

FIG. 5 is a block diagram that illustrates a configuration of an information terminal in the present example embodiment.

FIG. 6 is a flow chart that illustrates a flow of a medical checkup presentation operation performed by the medical checkup presentation server.

FIG. 7 conceptionally illustrates a detail of an operation of a medical checkup selection unit.

FIG. 8 conceptionally illustrates a detail of a learning operation for building a machine learning model.

FIG. 9 conceptionally illustrates a detail of an operation of the medical checkup selection unit using the machine learning model.

FIG. 10 illustrates one example of a recommended medical checkup information that is presented to a presentation target person.

FIG. 11 illustrates another example of the recommended medical checkup information that is presented to the presentation target person.

FIG. 12 illustrates one example of the recommended medical checkup information that is presented to a healthcare worker.

FIG. 13 illustrates one example of a recommended medical checkup information that is presented to a patient.

### DESCRIPTION OF EXAMPLE EMBODIMENTS

Next, an example embodiment of a medical checkup presentation apparatus, a medical checkup presentation method and a recording medium will be described with reference to the drawings. In the below described description, a medical checkup presentation system SYS to which the example embodiment of the medical checkup presentation apparatus, the medical checkup presentation method and the recording medium is applied will be described. The medical checkup presentation system SYS may be used in a healthcare institution where a healthcare service is provided to a patient, for example. The healthcare institution may include at least one of a hospital, a health clinic, a long-term care health facility, a dispensing pharmacy and any facility that provides the healthcare service (for example, at least one of a facility that provides a health check and a facility that provides any clinical test), for example.

(1) Configuration of Medical Checkup Presentation System SYS

Firstly, a configuration of the medical checkup presentation system SYS will be described.

(1-1) Entire Configuration of Medical Checkup Presentation System SYS

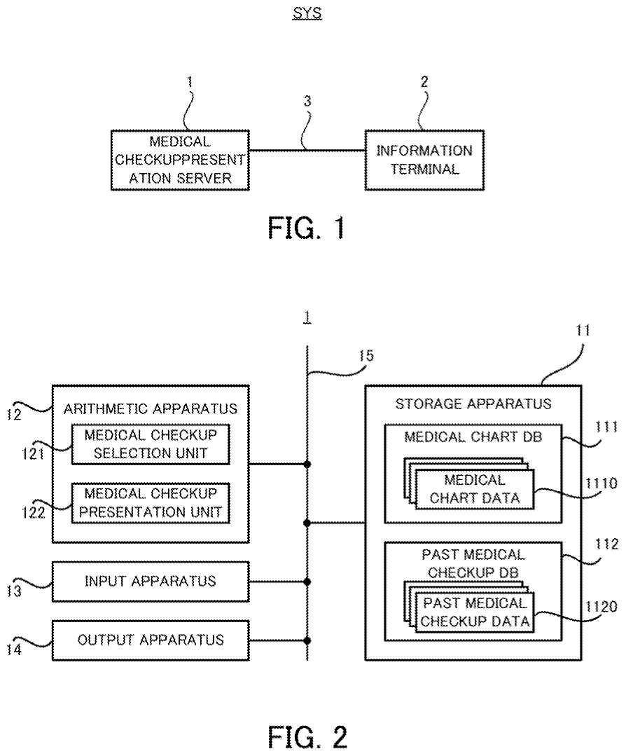

Firstly, with reference to FIG. 1, :an entire configuration of the medical checkup presentation system SYS in the present example embodiment will be described. FIG. 1 is a block diagram that illustrates the entire configuration of the medical checkup presentation system SYS in the present example embodiment.

The medical checkup presentation system SYS is configured to select a medical checkup recommended to a medical checkup target person (in the below described description, it is referred to as “a recommended medical checkup”) and to present, to a presentation target person, a recommended medical checkup information indicating at least an item name of the selected recommended medical checkup. The medical checkup presentation system SYS may propose that the presentation target person should take the recommended medical checkup by presenting the recommended medical checkup information to the presentation target person. The “medical checkup target person” in the present example embodiment is any person who is recommended to take the recommended medical checkup. The medical checkup target person may include a patient who has taken a medical examination in the healthcare institution that uses the medical checkup presentation system SYS, for example. Moreover, the “medical checkup” in the present example embodiment includes any medical checkup (for example, a clinical test) related to a healthcare. The clinical test may include a physiological test (in other words, a physiological function test) that directly tests a body of the medical checkup target person, and may include an antibody test that tests a sample (namely, a specimen material) collected from the body of the test target person. Moreover, the “presentation target person” in the present example embodiment is any person to whom the recommended medical checkup information is presented. The presentation target person may be a person who is same as the medical checkup target person. The presentation target person may be a person (for example, a healthcare worker) who is different from the medical checkup target person. Note that the “healthcare worker” in the present example embodiment is a person in general engaged in a healthcare, which is an activity for the purpose of at least one of a treatment of a disease, a prevention of the disease, a maintenance of a health, a restoration of a health, and an improvement of a health. The healthcare worker may include at least one person of a doctor, a dentist, a healthcare professional (for example, at least one person of a nurse, a pharmacist, a clinical laboratory technician, a radiology technician, a physical therapist and so on) and so on.

In order to select the recommended medical checkup and present the recommended medical checkup information indicating the selected recommended medical checkup to the presentation target person, the medical checkup presentation system SYS includes a medical checkup presentation server 1 that is one specific example of “a medical checkup presentation apparatus” and an information terminal 2 that is one specific example of “a presentation device”, as illustrated in FIG. 1. The medical checkup presentation server 1 and the information terminal 2 are configured to communicate with each other through a communication network 3. The communication network 3 may include a wired communication network and may include a wireless communication network.

The medical checkup presentation server 1 is an information processing apparatus that is usable by the healthcare institution at which the medical checkup target person takes a medical examination. Thus, the medical checkup presentation server 1 is typically placed for the healthcare institution. In this case, the medical checkup presentation server 1 may be placed inside the healthcare institution (for example, inside a healthcare facility of the healthcare institution), and may be placed outside the healthcare institution (for example, a facility placed outside the healthcare facility of the healthcare institution). The medical checkup presentation server 1 is configured to perform a medical checkup presentation operation. Specifically, the medical checkup presentation server 1 may perform, as a part of the medical checkup presentation operation, an operation for selecting the recommended medical checkup. Furthermore, the medical checkup presentation server 1 may perform, as another part of the medical checkup presentation operation, an operation for presenting the recommended medical checkup information to the presentation target person by using the information terminal 2 that is configured to present any information to the presentation target person. Namely, the medical checkup presentation server 1 may perform, as another part of the medical checkup presentation operation, an operation for controlling the information terminal 2 to present the recommended medical checkup information to the presentation target person.

The information terminal 2 is an information processing apparatus that is usable by the presentation target person. An information processing apparatus (for example, at least one of a personal computer and a tablet terminal) that is placed for the healthcare institution may be used as the information terminal 2. An information processing apparatus (for example, a tablet terminal) that is lent to the presentation target person by the healthcare institution may be used as the information terminal 2. An information processing apparatus (for example, at least one of a personal computer, a tablet terminal and a smartphone) of the presentation target person may be used as the information terminal 2. The information terminal 2 is configured to present the recommended medical checkup information to the presentation target person under the control of the medical checkup presentation server 1, as described above. As a result, the presentation target person can recognize the recommended medical checkup. Namely, the presentation target person can recognize what medical checkup is recommended (in other words, suggested or proposed) for the medical checkup target person to take

(1-2) Configuration of Medical Checkup Presentation Server 1

Next, with reference to FIG. 2, a configuration of the medical checkup presentation server 1 will be described. FIG. 2 is a block diagram that illustrates the configuration of the medical checkup presentation server 1.

As illustrated in FIG. 2, the medical checkup presentation server 1 includes a storage apparatus 11 and an arithmetic apparatus 12. Furthermore, the medical checkup presentation server 1 may include an input apparatus 13 and an output apparatus 14. However, the medical checkup presentation server 1 may not include at least one of the input apparatus 13 and the output apparatus 14. The storage apparatus 11, arithmetic apparatus 12, the input apparatus 13 and the output apparatus 14 may be interconnected through a data bus 15.

The storage apparatus 11 is configured to store desired data. For example, the storage apparatus 11 may temporarily store the computer program that is executed by the arithmetic apparatus 12. The storage apparatus 11 may temporarily store data temporarily used by the arithmetic apparatus 12 when the arithmetic apparatus 12 executes the computer program. The storage apparatus 11 may store data stored for a long term by the medical checkup presentation server 1. The storage apparatus 11 may include at least one of a RAM (Random Access Memory), a ROM (Read Only Memory), a hard disk apparatus, a magneto-optical disc, a SSD (Solid State Drive) and a disk array apparatus. Namely, the storage apparatus 11 may include a non-transitory recording medium.

In the present example embodiment, the storage apparatus 11 stores data used by the medical checkup presentation server 1 to select the recommended medical checkup. FIG. 2 illustrates a medical chart DB (Database) 111 and a past medical checkup DB 112 as examples of the data used by the medical checkup presentation server 1 to select the recommended medical checkup. Namely, FIG. 2 illustrates an example in which the storage apparatus 11 stores the medical chart DB 111 and the past medical checkup DB 112.

The medical chart DB 111 is a database that stores medical chart data 1110 of the patient who has taken the medical examination (namely, at least one of a medical consultation and the treatment) in the healthcare institution for which the medical checkup presentation server 1 is placed. Generally, there are a plurality of patients who have taken the medical examination in the healthcare institution. Thus, the medical chart DB 111 typically stores a plurality of medical chart data 1110 that correspond to the plurality of patients, respectively. Each medical chart data 1110 includes at least data related to a history of the medical examination that has been taken in the healthcare institution by one patient corresponding to each medical chart data 1110.

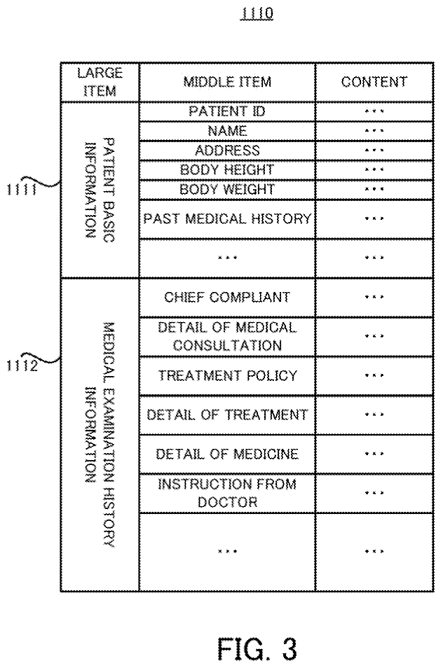

FIG. 3 illustrates one example of the medical chart data 1110. As illustrated in FIG. 3, the medical chart data 1110 may include a patient basic information 1111 and a medical examination history information 1112.

The patient basic information 1111 includes an information related to the individual patient (typically, a personal information). For example, in an example illustrated in FIG. 3, the patient basic information 1111 includes a patient ID for identifying the patient, an information related to a name of the patient, an information related to an address of the patient, an information related to a body height of the patient, an information related to a body weight of the patient, and an information related to a past medical history (for example, at least one of a history of a disease, a history of a medication and a history of an allergy) of the patient. However, the patient basic information 1111 may not include at least one of plurality of these information. The patient basic information 1111 may include other information in addition to or instead of at least one of the plurality of information illustrated in FIG. 3. For example, the patient basic information 1111 may include at least one of an information related to a birth date of the patient, an information related to a phone number of the patient, an information related to a place of a work of the patient, an information related to an emergency contact number of the patient, an information of a social history of the patient, an information related to a family history of the patient (namely, a past medical history of a family or a housemate of the patient) and an information related to a genome sequence of the patient.

The medical examination history information 1112 includes an information related to the history of the medical examination (in other words, a process of the medical examination) that has been taken by the patient in the healthcare institution for which the medical checkup presentation server 1 is placed. For example, in the example illustrated in FIG. 3, the medical examination history information 1112 includes an information related to a chief compliant, an information related to a detail of the medical consultation by the doctor, an information related to a treatment policy decided by the doctor, an information related to a detail of the treatment provided by the doctor, an information related to a detail of a medicine prescribed by the doctor, and an information related to a detail of an instruction from the doctor to the patient or the healthcare worker. However, the medical examination history information 1112 may not include at least one of the plurality of these information. The medical examination history information 1112 may include other information in addition to or instead of at least one of the plurality of information illustrated in FIG. 3. For example, the medical examination history information 1112 may include at least one of an information related to a history of an informed consent, an information related to an explanation and so on provided from the healthcare worker to the patient and an information related to a question and so on asked by the patient to the healthcare worker.

There is a possibility that the patient has taken the medical examination in another healthcare institution that is different from one healthcare institution, in addition to or instead of one healthcare institution for which the medical checkup presentation server 1 is placed. In this case, the medical chart data 1110 (especially, the medical examination history information 1112) may include an information related to the history of the medical examination that has been taken by the patient in another healthcare institution, in addition to or instead of the information related to the history of the medical examination that has been taken by the patient in one healthcare institution. For example, the medical chart data 1110 may include an information related to the history of the medical examination that has been taken by the patient in another healthcare institution affiliated with (namely, having same affiliation with) one healthcare institution. Alternatively, the information related to the history of the medical examination that has been taken by the patient in another healthcare institution may be stored in a storage apparatus placed for another healthcare institution in a form that allows the medical checkup presentation server 1 placed for one healthcare institution to use it. In this case, the medical checkup presentation server 1 may download or use, through a communication network, the information stored in the storage apparatus placed for another healthcare institution.

The past medical checkup DB 112 is a database that stores past medical checkup data 1120 including an information related to a history of the medical checkup that has checked in the past in the healthcare institution for which the medical checkup presentation server 1 is placed. Specifically, the past medical checkup DB 112 is a database that stores the past medical checkup data 1120 including the information related to the history of the medical checkup of a checked person who has taken the medical checkup in the past in the healthcare institution for which the medical checkup presentation server 1 is placed. Generally, there are a plurality of checked persons who have taken the medical checkup in the healthcare institution. Thus, the past medical checkup DB 112 typically stores a plurality of past medical checkup data 1120 that correspond to the plurality of checked persons, respectively. Each past medical checkup data 1120 includes at least an information related to the history of the medical checkup that has been taken in the healthcare institution by one checked person corresponding to each past medical checkup data 1120. Moreover, most of reasons why the checked person takes the medical checkup in the healthcare institution is a reason that the checked person takes the medical examination in the healthcare institution. Thus, the checked person is typically the patient who has taken the medical examination in the healthcare institution for which the medical checkup presentation server 1 is placed. However, the checked person may not be the patient who has taken the medical examination in the healthcare institution for which the medical checkup presentation server 1 is placed.

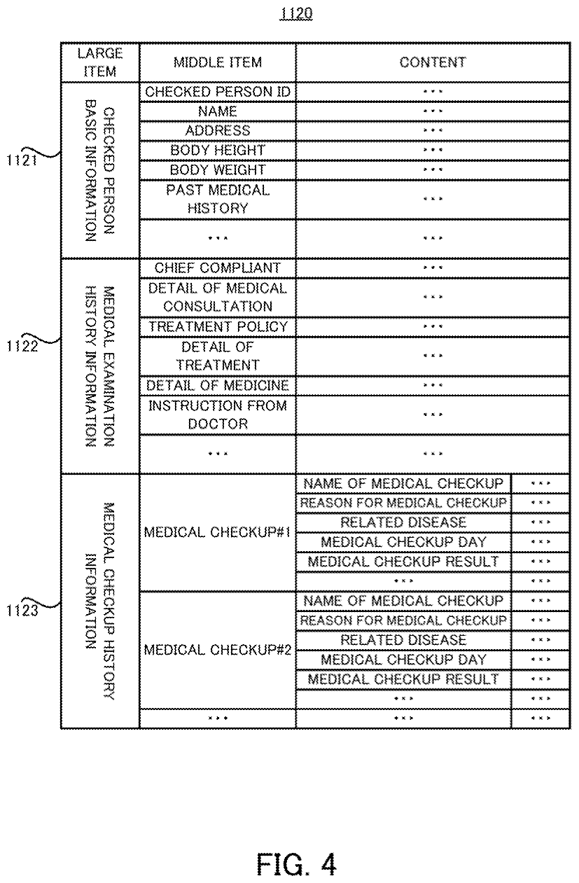

FIG. 4 illustrates one example of the past medical checkup data 1120. As illustrated in FIG. 4, the past medical checkup data 1120 may include a checked person basic information 1121, a medical examination history information 1122 and a medical checkup history information 1123. However, the past medical checkup data 1120 may not include at least one of the checked person basic information 1121 and the medical examination history information 1122.

The checked person basic information 1121 may be same as the patient basic information 1111, which includes the information related to the individual patient, in that it includes an information related to the individual checked person (typically, a personal information). Namely, a type (namely, an item) of the information included in the checked person basic information 1121 may be same as a type (namely, an item) of the information included in the patient basic information 1111 at least partially. For example, in an example illustrated in FIG. 4, the checked person basic information 1121 includes a checked person ID for identifying the checked person, an information related to the name of the checked person, an information related to the address of the checked person, an information related to the body height of the checked person, an information related to the body weight, and an information related to the past medical history of the checked person.

The medical examination history information 1122 may be same as the medical examination history information 1112, which includes the information related to the history of the medical examination that has been taken by the patient in the healthcare institution for which the medical checkup presentation server 1 is placed, in that it includes an information related to the history of the medical examination (in other words, a process of the medical examination) that has been taken by the checked person in the healthcare institution for which the medical checkup presentation server 1 is placed. Namely, a type (namely, an item) of the information included in the medical examination history information 1122 may be same as a type (namely, an item) of the information included in the medical examination history information 1112 at least partially. For example, in the example illustrated in FIG. 4, the medical examination history information 1122 includes an information related to a chief compliant, an information related to a detail of the medical consultation by the doctor, an information related to the treatment policy decided by the doctor, an information related to a detail of the treatment provided by the doctor, an information related to a detail of the medicine prescribed by the doctor, and an information related to a detail of the instruction from the doctor to the checked person or the healthcare worker.

When the checked person is the patient as described above, the checked person basic information 1121 and the medical examination history information 1122 included in the past medical checkup data 1120 may be same as the patient basic information 1111 and the medical examination history information 1112 included in the medical chart data 1110. In this case, the past medical checkup data 1120 may include pointer data for referencing the patient basic information 1111 and the medical examination history information 1112 included in the medical chart data 1110, instead of the checked person basic information 1121 and the medical examination history information 1122. In this case, at least a part of the medical chart data 1110 may be used as at least a part of the past medical checkup data 1120.

The medical checkup history information 1123 includes an information related to the history of the medical checkup that has been taken by the checked person in the healthcare institution for which the medical checkup presentation server 1 is placed. For example, in the example illustrated in FIG. 4, the medical checkup history information 1123 includes an information related to an item name (namely, a name) of the medical checkup, an information related to a reason why the medical checkup has been performed, an information related to a disease pertaining to the medical checkup (namely, a disease that is expected to be assessed by the medical checkup), an information related to a date on which the medical checkup was performed and an information related to a result of the medical checkup. However, the medical checkup history information 1123 may not include at least one of the plurality of these information. The medical checkup history information 1123 may include other information in addition to or instead of at least one of the plurality of information illustrated in FIG. 4.

There is a possibility that the checked person has taken multiple types of medical checkups. In this case, the medical checkup history information 1123 may include the information related to the history of each of the multiple types of medical checkups. For example, when the checked person has taken a first type of medical checkup and a second type of medical checkup, the medical checkup history information 1123 may include the information related to the history of the first type of medical checkup and the information related to the history of the second type of medical checkup.

There is a possibility that the checked person has taken the medical examination in another healthcare institution that is different from the healthcare institution for which the medical checkup presentation server 1 is placed, in addition to or instead of one healthcare institution for which the medical checkup presentation server 1 is placed. In this case, the past medical checkup data 1120 (especially, the medical examination history information 1122) may include an information related to the history of the medical examination that has been taken by the checked person in another healthcare institution, in addition to or instead of the information related to the history of the medical examination that has been taken by the checked person in one healthcare institution, as with the medical chart data 1110. Alternatively, the information related to the history of the medical examination that has been taken by the checked person in another healthcare institution may be stored in a storage apparatus placed for another healthcare institution in a form that allows the medical checkup presentation server 1 placed for one healthcare institution to use it. In this case, the medical checkup presentation server 1 may download or use, through a communication network, the information stored in the storage apparatus placed for another healthcare institution.

There is a possibility that the checked person has taken the medical checkup in another healthcare institution that is different from the healthcare institution for which the medical checkup presentation server 1 is placed, in addition to or instead of one healthcare institution for which the medical checkup presentation server 1 is placed. In this case, the medical chart data 1110 (especially, the medical checkup history information 1123) may include an information related to the history of the medical checkup that has been taken by the checked person in another healthcare institution, in addition to or instead of the information related to the history of the medical checkup that has been taken by the checked person in one healthcare institution. For example, the past medical checkup data 1120 may include an information related to the history of the medical checkup that has been taken by the checked person in another healthcare institution affiliated with (namely, having same affiliation with) one healthcare institution. Alternatively, the information related to the history of the medical checkup that has been taken by the checked person in another healthcare institution may be stored in a storage apparatus placed for another healthcare institution in a form that allows the medical checkup presentation server 1 placed for one healthcare institution to use it. In this case, the medical checkup presentation server 1 may download or use, through a communication network, the information stored in the storage apparatus placed for another healthcare institution.

Again in FIG. 2, the arithmetic apparatus 12 includes a CPU (Central Processing Unit), for example. The arithmetic apparatus 12 reads a computer program. For example, the arithmetic apparatus 12 may read a computer program stored in the storage apparatus 11. For example, the arithmetic apparatus 12 may read a computer program stored in a computer-readable and non-transitory recording medium, by using a not-illustrated recording medium reading apparatus. The arithmetic apparatus 12 may obtain (namely, download or read) a computer program from a not-illustrated apparatus placed outside the medical checkup presentation server 1 through a not-illustrated communication apparatus. The arithmetic apparatus 12 executes the read computer program. As a result, a logical functional block for performing an operation (for example, the above described medical checkup presentation operation) that should be performed by the medical checkup presentation server 1 is implemented in the arithmetic apparatus 12. Namely, the arithmetic apparatus 12 is configured to serve as a controller for implementing the logical block for performing the operation that should be performed by the medical checkup presentation server 1.

FIG. 2 illustrates one example of the logical block that is implemented in the arithmetic apparatus 12 to perform the medical checkup presentation operation. As illustrated in FIG. 2, a medical checkup selection unit 121 that is one specific example of “a selection unit” and a medical checkup presentation unit 122 that is one specific example of “a presentation unit” are implemented in the arithmetic apparatus 12. Note that a detail of the operation performed by the medical checkup selection unit 121 and the medical checkup presentation unit 122 will be described later in detail, however, an overview thereof is briefly described here. The medical checkup selection unit 121 is configured to select the recommended medical checkup based on the medical chart DB 111 and the past DB 112. The medical checkup presentation unit 122 is configured to control the information terminal 2 to present, to the presentation target, the recommended medical checkup information indicating at least the item name of the recommended medical checkup selected by the medical checkup selection unit 121.

The input apparatus 13 is an apparatus that is configured to receive an input of an information from an outside of the medical checkup presentation server 1 to the medical checkup presentation server 1.

The output apparatus 14 is an apparatus that is configured to output an information to an outside of the medical checkup presentation server 1. For example, the output apparatus 14 may output an information related to the medical checkup presentation operation performed by the medical checkup presentation server 1. For example, the output apparatus 14 may output (namely, transmit), to the information terminal 2 through the communication network 3, a control information for controlling the information terminal 2 to present the recommended medical checkup information.

(1-3) Configuration of Information Terminal 2

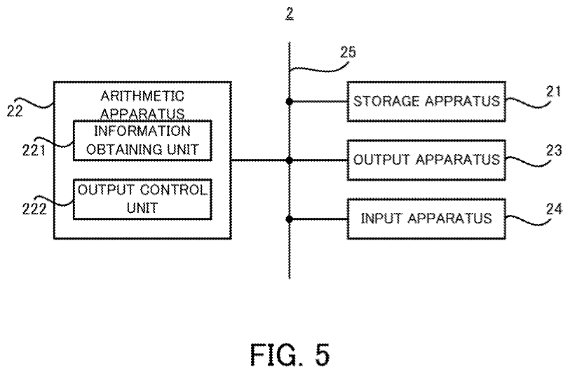

Next, with reference to FIG. 5, a configuration of the information terminal 2 will be described. FIG. 5 is a block diagram that illustrates the configuration of the information terminal 2.

As illustrated in FIG. 5, the information terminal 2 includes a storage apparatus 21, an arithmetic apparatus 22 and an output apparatus 23. Furthermore, the information terminal 2 may include an input apparatus 24. However, the information terminal 2 may not include the input apparatus 24. The storage apparatus 21, arithmetic apparatus 22, the output apparatus 23 and the input apparatus 24 may be interconnected through a data bus 25.

The storage apparatus 21 is configured to store desired data. For example, the storage apparatus 21 may temporarily store the computer program that is executed by the arithmetic apparatus 22. The storage apparatus 21 may temporarily store data temporarily used by the arithmetic apparatus 22 when the arithmetic apparatus 22 executes the computer program. The storage apparatus 21 may store data stored for a long term by the information terminal 2. The storage apparatus 21 may include at least one of a RAM, a ROM, a hard disk apparatus, a magneto-optical disc, a SSD and a disk array apparatus. Namely, the storage apparatus 21 may include a non-transitory recording medium.

The arithmetic apparatus 22 includes a CPU, for example. The arithmetic apparatus 22 reads a computer program. For example, the arithmetic apparatus 22 may read a computer program stored in the storage apparatus 21. For example, the arithmetic apparatus 22 may read a computer program stored in a computer-readable and non-transitory recording medium, by using a not-illustrated recording medium reading apparatus. The arithmetic apparatus 22 may obtain (namely, download or read) a computer program from a not-illustrated apparatus placed outside the information terminal 2 through a not-illustrated communication apparatus. The arithmetic apparatus 22 executes the read computer program. As a result, a logical functional block for performing an operation that should be performed by the information terminal 2 is implemented in the arithmetic apparatus 22. Namely, the arithmetic apparatus 22 is configured to serve as a controller for implementing the logical block for performing the operation that should be performed by the information terminal 2.

In the present example embodiment, the information terminal 2 is configured to perform a recommended medical checkup information output operation for outputting the recommended medical checkup information in a form that allows the presentation target person to recognize it, under the control the medical checkup presentation server 1. FIG. 5 illustrates one example of the logical block that is implemented in the arithmetic apparatus 22 to perform the recommended medical checkup information output operation. As illustrated in FIG. 5, an information obtaining unit 221 and an output control unit 222 are implemented in the arithmetic apparatus 22. The information obtaining unit 221 is configured to obtain the control information for controlling the information terminal 2 to present the recommended medical checkup information. The output control unit 222 is configured to control, based on the control information obtained by the information obtaining unit 221, the output apparatus 23 to output recommended medical checkup information in a form that allows the presentation target person to recognize it.

The output apparatus 23 is an apparatus that is configured to output an information to an outside of the information terminal 2. For example, the output apparatus 23 may output an image. Namely, the output apparatus 23 may include a display apparatus (what we call a display) that is configured to display the image. In this case, the output apparatus 23 may present the recommended medical checkup information to the presentation target person by displaying the recommended medical checkup information as the image. As a result, the presentation target person can recognize the recommended medical checkup information by using a visual sense. For example, the output apparatus 23 may output a sound. Namely, the output apparatus 23 may include a sound apparatus (what we call a speaker) that is configured to output the sound. In this case, the output apparatus 23 may present the recommended medical checkup information to the presentation target person by outputting the recommended medical checkup information as the sound. As a result, the presentation target person can recognize the recommended medical checkup information by using a hearing sense. For example, the output apparatus 23 may output the information on a paper. Namely, the output apparatus 23 may include a print apparatus (what we call a printer) that is configured to print a desired information on the paper. In this case, the output apparatus 23 may present the recommended medical checkup information to the presentation target person by printing the recommended medical checkup information on the paper. As a result, the presentation target person can recognize the recommended medical checkup information by using the visual sense.

The input apparatus 24 is an apparatus that is configured to receive an input of an information from an outside of the information terminal 2 to the information terminal 2.

A presentation method of the recommended medical checkup information using the information terminal 2 may be changed depending on the presentation target person under the control of the arithmetic apparatus 22. For example, the presentation method of the recommended medical checkup information may include a place at which the recommended medical checkup information is presented. As one specific example, the information terminal 2 may be placed on a traffic line of the presentation target person in the healthcare institution for which the medical checkup presentation server 1 is placed. For example, when the presentation target person is the medical checkup target person (for example, the patient) as described above, the information terminal 2 may be placed on the traffic line of the patient in the healthcare institution. The traffic line of the patient in the healthcare institution may include a traffic line between a reception, a waiting room and a medical consultation room in the healthcare institution. As one example, the information terminal 2 may be placed at the reception of the healthcare institution (for example, a place where the patient who has finished taking the medical examination pays a healthcare expenditure). In this case, the patient can recognize the recommended medical checkup selected based on the history of the medical examination at a timing when he pays the healthcare expenditure after taking the medical examination in the healthcare examination. As another example, the information terminal 2 may be placed in the waiting room of the healthcare institution (for example, a place where the patient wait for the medical examination or the payment of the healthcare expenditure). In this case, the patient can recognize the recommended medical checkup selected based on the history of the medical examination at a timing when he waits for the payment of the healthcare expenditure after taking the medical examination in the healthcare examination. As another specific example, when the presentation target person is the healthcare worker as described above, the information terminal 2 may be placed in a room where the medical examination is performed (for example, the medical consultation room). In this case, the healthcare worker can recognize the recommended medical checkup while performing the medical examination for the patient (namely, the medical checkup target person). As a result, the healthcare worker can inform the patient of the recommended medical checkup while performing the medical examination for the patient (namely, the medical checkup target person).

Alternatively, when the information processing apparatus of the medical checkup target person is used as the information terminal 2 as described above, the medical checkup target person can recognize the recommended medical checkup, which is selected based on the history of the medical examination, at a desired place (for example, a home of the medical checkup target person and the like) without vising the healthcare institution.

However, both of the healthcare worker and the patient may recognize the recommended medical checkup by using the same information terminal 2. For example, when the information terminal 2 is placed in the room where the medical examination is performed, both of the healthcare worker and the patient may recognize the recommended medical checkup by using the information terminal 2. Namely, the presentation method of the recommended medical checkup information using the information terminal 2 may not be changed depending on the presentation target person. However, there is a possibility that a part of the information that is allowed to be disclosed to the healthcare worker should not be disclosed to the patient because of a legal constraint. Thus, when both of the healthcare worker and the patient recognizes the recommended medical checkup by using the same information terminal 2, the information terminal 2 may present the recommended medical checkup information not including the information that should not be disclosed to the patient under the control of the medical checkup presentation unit 122. Alternatively, the information terminal 2 may perform a switching between a presentation content when the recommended medical checkup information is presented to the patient and a presentation content when the recommended medical checkup information is presented to the healthcare institution. For example, when the information terminal 2 includes the display as the output apparatus 23, the information terminal 2 may perform a switching between a display screen used to present to the patient the recommended medical checkup information that does not include the information that should not be disclosed to the patient and a display screen used to present to the healthcare worker the recommended medical checkup information that may include the information that should not be disclosed to the patient.

(2) Medical Checkup Presentation Operation Performed by Medical Checkup Presentation Server 1

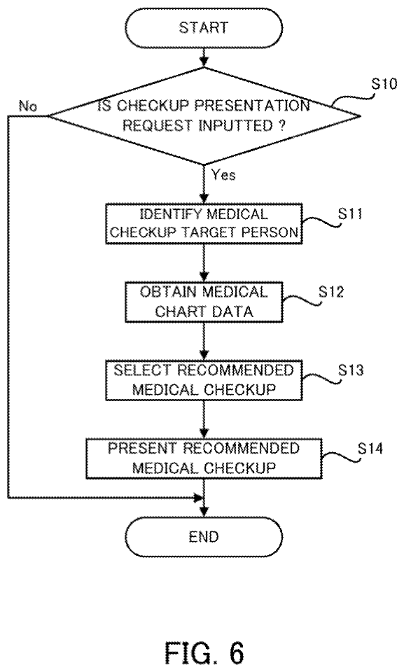

Next, with reference to FIG. 6, the medical checkup presentation operation performed by the medical checkup presentation server 1 will be described. FIG. 6 is a flowchart that illustrates the medical checkup presentation operation performed by the medical checkup presentation server 1.

As illustrated in FIG. 6, firstly, the medical checkup selection unit 121 determines whether or not the medical checkup presentation request, which is to request the medical checkup presentation server 1 to perform the medical checkup presentation operation, is inputted into the medical checkup presentation server 1 (a step S10). The medical checkup presentation request may be inputted into the medical checkup presentation server 1 by at least one of the presentation target person and the medical checkup target person. For example, when the presentation target person is same as the medical checkup target person, the presentation target person (namely, the medical checkup target person) may transmit (namely, input) the medical checkup presentation request to the medical checkup presentation server 1 by using the input apparatus 24 of the information terminal 2. For example, when the presentation target person is a person (for example, the healthcare worker) who is different from the medical checkup target person, the presentation target person (for example, the healthcare worker) may transmit (namely, input) the medical checkup presentation request to the medical checkup presentation server 1 by using the input apparatus 13 of the medical checkup presentation server 1.

As a result of the determination at the step S10, when it is determined that the medical checkup presentation request is not inputted (the step S10: No), the medical checkup presentation server 1 may end the medical checkup presentation operation illustrated in FIG. 6. After ending the medical checkup presentation operation, the medical checkup presentation server 1 may star the medical checkup presentation operation again after a predetermined time elapses.

On the other hand, as a result of the determination at the step S10, when it is determined that the medical checkup presentation request is inputted (the step S10: Yes), the medical checkup selection unit 121 selects the recommended medical checkup (a step S11 to a step S13). Specifically, the medical checkup selection unit 121 firstly identifies the medical checkup target person based on an information included in the medical checkup presentation request (the step S11). Thus, it is preferable that the medical checkup presentation request include an information that is capable of identifying the medical checkup target person. Alternatively, the medical checkup selection unit 121 may obtain the information that is capable of identifying the medical checkup target person separately from the medical checkup presentation request. Then, the medical checkup selection unit 121 obtains, from the medical chart DB 111 stored in the storage apparatus 11, the medical chart data 1110 corresponding to the medical checkup target person identified at the step S11 (the step S12). Then, the medical checkup selection unit 121 selects the recommended medical checkup based on the medical chart data 1110 obtained at the step S12 and the past DB 112 stored in the storage apparatus 11 (the step S13).

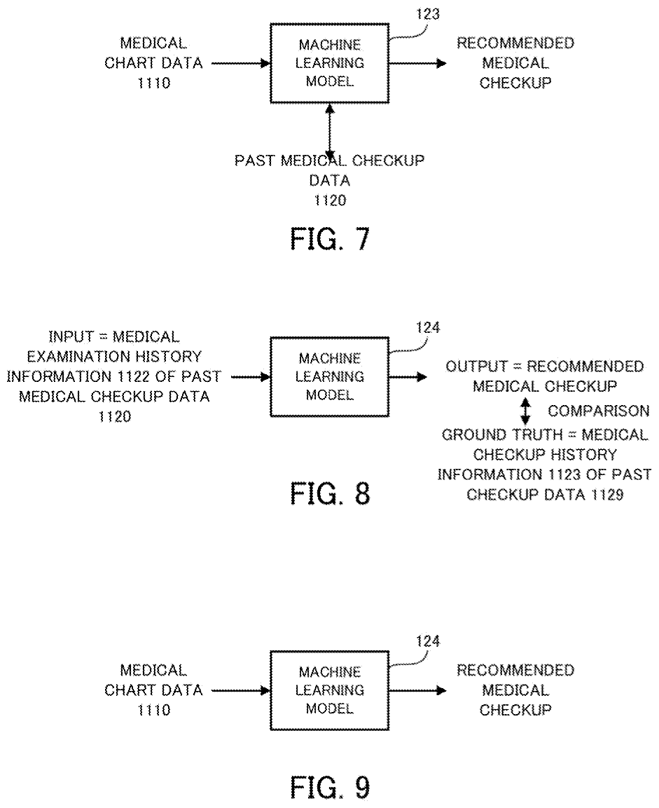

For example, the medical checkup selection unit 121 may determine, as the recommended medical checkup, at least a part of the medical checkup that has been taken by the checked person who corresponds to the medical examination history information 1122 at least a part of which is same as or similar to at least a part of the medical examination history information 1112 of the medical checkup target person. Namely, the medical checkup selection unit 121 may select, as the recommended medical checkup, at least a part of the medical checkup that has been taken by the checked person who corresponds to the history of the medical examination at least a part of which is same as or similar to that of the medical checkup target person. In this case, as illustrated in FIG. 7 that conceptionally illustrates a detail of the operation of the medical checkup selection unit 121, the medical checkup selection unit 121 may select the recommended medical checkup by using a selection engine 123 that is configured to output the recommended medical checkup by searching the plurality of past medical checkup data 1120 based on the medical chart data 1110 when the medical chart data 1110 of the medical checkup target person is inputted thereto. The selection engine 123 may be a rule-based engine that selects, as the recommended medical checkup, at least a part of the medical checkup that has been taken by the checked person who corresponds to the history of the medical examination at least a part of which is same as or similar to that of the medical checkup target person.

Alternatively, for example, the medical checkup selection unit 121 may select the recommended medical checkup by using a machine learning model 124 that is configured to output an item name of the recommended medical checkup when the medical chart data 1110 of the medical checkup target person is inputted thereto. Note that the machine learning model 124 may be a machine learning model using a Neural Network, for example. In this case, it is preferable to perform a learning operation for setting a parameter of the machine learning model 124 (for example, at least one of a weight and a bias of the Neural Network) by using the plurality of past medical checkup data 1120 stored in the past medical checkup DB 112, before the medical checkup selection unit 121 selects the recommended medical checkup by using the machine learning model 124. Specifically, as illustrated in FIG. 8 that conceptionally illustrates a detail of the learning operation for building the machine learning model 124, the learning operation may be an operation for setting the parameter of the machine learning model 124 so that a difference between an information related to the recommended medical checkup outputted from the machine learning model 124 when at least the medical examination history information 1122 included in the past medical checkup data 1120 is inputted into the machine learning model 124 and an information included in the medical checkup history information 1123 included in the past medical checkup data 1120 is smaller. For example, the learning operation may be an operation for setting the parameter of the machine learning model 124 so that a difference between the item name of the recommended medical checkup outputted from the machine learning model 124 and the item name of the medical checkup included in the medical checkup history information 1123 included in the past medical checkup data 1120 is smaller. After the learning operation of the machine learning model 124 is completed, the medical checkup selection unit 121 may select the recommended medical checkup by inputting the medical chart data 1110 of the medical checkup target person into the machine learning model 124, as illustrated in FIG. 9 that conceptionally illustrates a detail of the operation of the medical checkup selection unit 121. Even in this case, it can be said that the medical checkup selection unit 121 selects the recommended medical checkup based on the past medical checkup data 1120, because it selects the recommended medical checkup by using the machine learning model 124 built based on the past medical checkup data 1120. Note that the medical checkup selection unit 121 may not use the past medical checkup DB 112 in order to select the recommended medical checkup using the machine learning model 124. Thus, the storage apparatus 11 may not necessarily store the past medical checkup DB 112.

The medical checkup selection unit 121 may select the recommended medical checkup by another method in addition to or instead of selecting, as the recommended medical checkup, at least a part of the medical checkup that has been taken by the checked person who corresponds to the history of the medical examination at least a part of which is same as or similar to that of the medical checkup target person

For example, the medical checkup selection unit 121 may select, as the recommended medical checkup, at least a part of the medical checkup that has been taken by the checked person who corresponds to the checked person basic information 1121 at least a part of which is same as or similar to at least a part of the patient basic information 1111 of the medical checkup target person. As one example, the medical checkup selection unit 121 may select, as the recommended medical checkup, at least a part of the medical checkup that has been taken by the checked person who corresponds to the past medical history at least a part of which is same as or similar to that of the medical checkup target person. As another example, the medical checkup selection unit 121 may select, as the recommended medical checkup, at least a part of the medical checkup that has been taken by the checked person who corresponds to the past family history at least a part of which is same as or similar to that of the medical checkup target person. As one example, the medical checkup selection unit 121 may select, as the recommended medical checkup, at least a part of the medical checkup that has been taken by the checked person who corresponds to the genome sequence at least a part of which is same as or similar to that of the medical checkup target person.

For example, the medical checkup selection unit 121 may select, as the recommended medical checkup, at least a part of the medical checkup that has been taken by the checked person who corresponds to a result of the health check at least a part of which is same as or similar to that of the medical checkup target person. In this case, it is preferable that the medical chart data 1110 include the information related to the result of the health check taken by the patient and it is preferable that the past medical checkup data 1120 include the information related to the result of the health check taken by the checked person. Note that the health check in the present example embodiment may mean a medical checkup performed for assessing a health condition of the patient at a stage when the disease is not developed yet or the patient does not aware of a sideration of the disease.

For example, the medical checkup selection unit 121 may select, as the recommended medical checkup, at least a part of the medical checkup that has been taken by the checked person who corresponds to a result of a medical interview at least a part of which is same as or similar to that of the medical checkup target person. In this case, it is preferable that the medical chart data 1110 include the information related to the result of the medical interview taken by the patient from the healthcare worker and it is preferable that the past medical checkup data 1120 include the information related to the result of the medical interview taken by the checked person from the healthcare worker.

For example, the medical checkup selection unit 121 may select, as the recommended medical checkup, at least a part of the medical checkup that has been taken by the checked person who corresponds to a life log (PHR: Personal Health Record) at least a part of which is same as or similar to that of the medical checkup target person. In this case, it is preferable that the medical chart data 1110 include the information related to the life log of the patient and it is preferable that the past medical checkup data 1120 include the information related to the life log of the checked person. Note that the “life log” in the present example embodiment may mean data that is obtained by recording at least one of a life and an action of a human in a chronological order. Typically, the “life log” in the present example embodiment may mean data that is obtained by recording a variation of the health condition of the human in the chronological order.

For example, the medical checkup selection unit 121 may select, as the recommended medical checkup, at least a part of the medical checkup that has been taken by the checked person who corresponds to an information related to the medical checkup that has been performed in the past at least a part of which is same as or similar to that of the medical checkup target person. As one example, the medical checkup selection unit 121 may select, as the recommended medical checkup, at least a part of the medical checkup that has been taken by the checked person who has taken the medical checkup in the past the item (the type) of at least a part of which is same as or similar to that of the medical checkup target person. As another example, the medical checkup selection unit 121 may select, as the recommended medical checkup, at least a part of the medical checkup that has been taken by the checked person who has taken the medical checkup in the past the result of at least a part of which is same as or similar to that of the medical checkup target person. In this case, it is preferable that the medical chart data 1110 include an information related to the history of the medical checkup that has been taken by the patient in the past (namely, an information that is similar to the medical checkup history information 1123 included in the past medical checkup data 1120).

For example, the medical checkup selection unit 121 may select the recommended medical checkup based on an information related to at least one of a weather and a climate at an residential place of the medical checkup target person. For example, when the weather at the residential place of the medical checkup target person is a weather that may cause a particular disease, the medical checkup for assessing this particular disease may be selected as the recommended medical checkup. For example, when the climate at the residential place of the medical checkup target person is a climate that may cause a particular disease, the medical checkup for assessing this particular disease may be selected as the recommended medical checkup.

For example, the medical checkup selection unit 121 may select the recommended medical checkup based on an information related to the residential place of the medical checkup target person. For example, when particular is prevalent or is likely to be prevalent at the residential place of the medical checkup target person, the medical checkup for assessing this particular disease may be selected as the recommended medical checkup.

For example, the medical checkup selection unit 121 may select the recommended medical checkup based on an information related to a medical reference. For example, the medical checkup selection unit 121 may analyze the medical reference and select the recommended medical checkup based on an analyzed result.

For example, the medical checkup selection unit 121 may select the recommended medical checkup so that a particular medical checkup, which is recommended by the healthcare institution itself for which the medical checkup presentation server 1 is placed, is more likely selected as the recommended medical checkup than another medical checkup other than the particular medical checkup. For example, when the healthcare institution focuses on the prevention or an early discovery of a particular disease, the medical checkup selection unit 121 may select the recommended medical checkup so that the particular medical checkup for assessing the particular disease is more likely selected as the recommended medical checkup than another medical checkup other than the particular medical checkup. For example, when the healthcare institution is aware of a presence of the particular medical checkup the importance of which is high but which is not taken by so many persons, the medical checkup selection unit 121 may select the recommended medical checkup so that the particular medical checkup is more likely selected as the recommended medical checkup than another medical checkup other than the particular medical checkup. For example, when the healthcare institution recognizes that a profit ratio of the particular medical checkup is relatively high, the medical checkup selection unit 121 may select the recommended medical checkup so that the particular medical checkup is more likely selected as the recommended medical checkup than another medical checkup other than the particular medical checkup. In this case, it is preferable that the past medical checkup DB 112 that is used to select the recommended medical checkup include an information related to the particular medical checkup that is recommended by the healthcare institution itself for which the medical checkup presentation server 1 is placed.

The medical checkup selection unit 121 may select each of a plurality of medical checkups as the recommended medical checkup. Namely, the medical checkup selection unit 121 may select a plurality of recommended medical checkups. For example, when there is a plurality of checked person who correspond to the history of the medical examination at least a part of which is same as or similar to that of the medical checkup target person, a plurality of medical checkups that has been taken by the plurality of checked persons, respectively, may be selected as the recommended medical checkups. For example, when the checked person, who corresponds to the history of the medical examination at least a part of which is same as or similar to that of the medical checkup target person, has taken a plurality of medical checkups, the plurality of medical checkups that have been taken by the checked person may be selected as the recommended medical checkups.

The medical checkup selection unit 121 may determine a reason why the recommended medical checkup is recommended to the medical checkup target person, in addition to selecting the recommended medical checkup. For example, when the medical checkup history information 1123 of the past medical checkup data 1120 includes the information related to the reason why the medical checkup has been performed as described above, the medical checkup selection unit 121 may determine, as the reason why the recommended medical checkup is recommended to the medical checkup target person, at least a part of the reason why the checked person, who corresponds to the history of the medical examination at least a part of which is same as or similar to that of the medical checkup target person, has taken the medical checkup.

The medical checkup selection unit 121 may determine an availability of an insurance for the recommended medical checkup, in addition to selecting the recommended medical checkup.

The medical checkup selection unit 121 may determine a disease that is expected to be assessed (in other words, that is expected to be checked) by the recommended medical checkup, in addition to selecting the recommended medical checkup. For example, when the medical checkup history information 1123 of the past medical checkup data 1120 includes the information related to the disease pertaining to the medical checkup, the medical checkup selection unit 121 may determine, as the disease that is expected to be assessed by the recommended medical checkup, at least a part of the disease pertaining to the medical checkup that has been taken by the checked person who corresponds to the history of the medical examination at least a part of which is same as or similar to that of the medical checkup target person.

The medical checkup selection unit 121 may calculate a degree of a risk (typically, a risk that is derived from a medical perspective) that the medical checkup target person catches the disease that is expected to be assessed by the recommended medical checkup, in addition to selecting the recommended medical checkup. The “risk that the medical checkup target person catches the disease” here may include a risk that the medical checkup target person, who has never caught the disease, catches the disease, and may include a risk that the medical checkup target person, who has caught the disease, catches the disease again (what we call a risk of a recurrence). For example, the medical checkup selection unit 121 may calculate (namely, estimate), as the degree of the risk that the medical checkup target person catches the disease that is expected to be assessed by the recommended medical checkup, a risk that the checked person catches the disease that is expected to be assessed by the recommended medical checkup based on the medical examination history information 1122 and so on of the checked person who corresponds to the history of the medical examination at least a part of which is same as or similar to that of the medical checkup target person.

The medical checkup selection unit 121 may determine a date on which the recommended medical checkup is recommended to be performed, in addition to selecting the recommended medical checkup. For example, when the medical checkup history information 1123 of the past medical checkup data 1120 includes the information related to the date on which the medical checkup was performed as described above, the medical checkup selection unit 121 may determine the date on which the medical checkup target person is recommended to take the recommended medical checkup based on the date on which the checked person, who corresponds to the history of the medical examination at least a part of which is same as or similar to that of the medical checkup target person, has taken the medical checkup.

The medical checkup selection unit 121 may calculate a degree of an recommendation of the recommended medical checkup, in addition to selecting the recommended medical checkup. Incidentally, in the present example embodiment, the degree of the recommendation of the recommended medical checkup may be distinguished from the above described degree of the risk, which may be an index for evaluating whether or not the recommended medical checkup is recommended from the medical perspective, in that it is an index for evaluating whether or not the recommended medical checkup is recommended from a viewpoint that is different from the medical perspective. For example, the medical checkup selection unit 121 may calculate the degree of the recommendation of the recommended medical checkup so that the degree of the recommendation of the medical checkup a cost of which is relatively high is smaller than the degree of the recommendation of the medical checkup a cost of which is relatively low. For example, the medical checkup selection unit 121 may calculate the degree of the recommendation of the recommended medical checkup so that the degree of the recommendation of the medical checkup for which the medical checkup target person needs to pay a relatively high out-of-pockets cost is smaller than the degree of the recommendation of the medical checkup for which the medical checkup target person needs to pay a relatively low out-of-pockets cost. For example, the medical checkup selection unit 121 may calculate the degree of the recommendation of the recommended medical checkup based on the address of the medical checkup target person so that the degree of the recommendation of the medical checkup that is necessary to be performed in a medical checkup facility that is relatively far from the address of the medical checkup target person is smaller than the degree of the recommendation of the medical checkup that is necessary to be performed in a medical checkup facility that is relatively close to the address of the medical checkup target person.

The medical checkup selection unit 121 may select a non-recommended medical checkup that is not recommended to be taken by the medical checkup target person, in addition to selecting the recommended medical checkup. For example, the medical checkup selection unit 121 may select, as the non-recommended medical checkup, the medical checkup that has been rarely taken by the checked person who corresponds to the history of the medical examination at least a part of which is same as or similar to that of the medical checkup target person. For example, the medical checkup selection unit 121 may select, as the non-recommended medical checkup, the medical checkup, which has been taken by the checked person who corresponds to the history of the medical examination at least a part of which is same as or similar to that of the medical checkup target person, but the result of which does not contribute the subsequent medical examination. Furthermore, the medical checkup selection unit 121 may determine a reason why the non-recommended medical checkup is not recommended together.

There is a higher possibility that the medical checkup target person takes the recommended medical checkup selected by the medical checkup selection unit 121. Thus, the medical checkup selection unit 121 may add an information related to the selected recommended medical checkup and so on to the past medical checkup data 1120 (especially, the medical checkup history information 1123) corresponding to the medical checkup target person. Namely, the medical checkup selection unit 121 may update the past medical checkup data 1120 (especially, the medical checkup history information 1123) corresponding to the medical checkup target person by using the information related to the selected recommended medical checkup and so on. Moreover, when the recommended medical checkup is selected by using the machine learning model 124 as described above, the learning operation for the machine learning model 124 may be performed again by using the updated past medical checkup data 1120. Namely, the output from the machine learning model 124 may be used as learning data for performing the operation for the machine learning model 124.

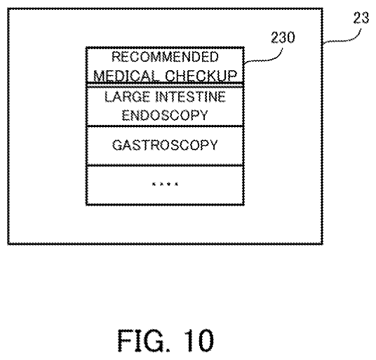

Then, the medical checkup presentation unit 122 controls the information terminal 2 to present, to the presentation target person, the recommended medical checkup information indicating at least the item name of the recommended medical checkup selected by the medical checkup selection unit 121 at the step S13 (a step S14). As a result, the information terminal 2 outputs, by using the output apparatus 23, the recommended medical checkup information in the form that allows the presentation target person to recognize it. For example, when the output apparatus 23 is the display apparatus, the output apparatus 23 may display, as the recommended medical checkup information, a GUI (Graphical User Interface) including a display field 230 indicating the item name of the recommended medical checkup, as illustrated in FIG. 10 that illustrates one example of the recommended medical checkup information presented to the presentation target person.

The medical checkup presentation unit 122 may start the operation at the step S14 in response to an output request for requesting the output apparatus 23 to actually output the recommended medical checkup information being inputted into the medical checkup presentation unit 122. The output request may be inputted into medical checkup presentation server 1 by at least one of the presentation target person and the medical checkup target person. For example, when the presentation target person is same as the medical checkup target person, the presentation target person (namely, the medical checkup target person) may transmit (namely, input) the output request to the medical checkup presentation server 1 by using the input apparatus 24 of the information terminal 2. For example, when the presentation target person is a person (for example, the healthcare worker) who is different from the medical checkup target person, the presentation target person (for example, the healthcare worker) may transmit (namely, input) the output request to the medical checkup presentation server 1 by using the input apparatus 13 of the medical checkup presentation server 1.

The output request may include a direct output request used by at least one of the presentation target person and the medical checkup target person to directly (in other words, actively) request the output apparatus 23 to actually output the recommended medical checkup information. The output request may include a direct output request used by at least one of the presentation target person and the medical checkup target person to indirectly (in other words, implicitly or non-actively) request the output apparatus 23 to actually output the recommended medical checkup information. For example, when the patient recognizes the recommended medical checkup at the timing when he pays the healthcare expenditure in the healthcare examination as described above, an operation for the payment of the healthcare expenditure may be used as the indirect output request.

When the medical checkup selection unit 121 selects the plurality of recommended medical checkups as described above, the medical checkup presentation unit 122 may control the information terminal 2 to present, to the presentation target person, the recommended medical checkup information indicating the item name of each of the plurality of recommended medical checkups. For example, the medical checkup presentation unit 122 may control the information terminal 2 to present the item names of the plurality of recommended medical checkups in a list form (namely, to display a list of the item names of the plurality of recommended medical checkups). For example, the medical checkup presentation unit 122 may control the information terminal 2 to present the item names of the plurality of recommended medical checkups in a pop-up form (namely, to display thumbnails of the item names of the plurality of recommended medical checkups and to enlarge the item name of at least one recommended medical checkup selected by the presentation target person). FIG. 10 illustrates one example of the recommended medical checkup information that is displayed in the list form when a large intestine endoscopy and a gastroscopy are selected as the recommended medical checkups.

As described above, the medical checkup selection unit 121 may determine an additional information related to the recommended medical checkup, in addition to selecting the recommended medical checkup. Note that an information related to at least one of the reason why the recommended medical checkup is recommended, the availability of the insurance for the recommended medical checkup, the disease that is expected to be assessed by the recommend medical checkup, the degree of the risk that the medical checkup target person catches the disease that is expected to be assessed by the recommended medical checkup, the date on which the recommended medical checkup is recommended to be performed, the degree of the recommendation of the recommended medical checkup and the non-recommended medical checkup as described above is one example of the additional information, for example. In this case, the medical checkup presentation unit 122 may control the information terminal 2 to present, to the presentation target person, the recommended medical checkup information including the additional information as described later.

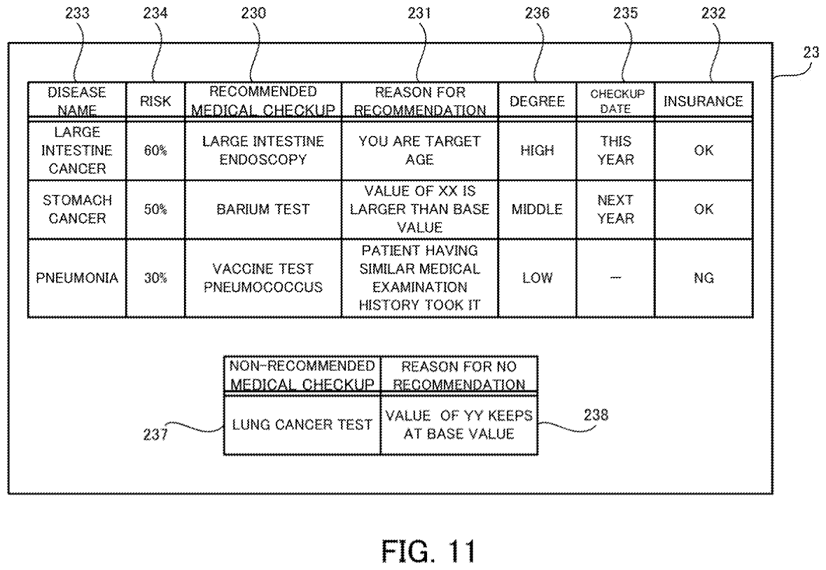

For example, when the medical checkup selection unit 121 determines, as the additional information, the reason why the recommended medical checkup is recommended to the medical checkup target person as describe above, the medical checkup presentation unit 122 may control the information terminal 2 to present, to the presentation target person, the recommended medical checkup information indicating not only the item name of the recommended medical checkup but also the reason why the recommended medical checkup is recommended to the medical checkup target person. For example, as illustrated in FIG. 11 that illustrates one example of the recommended medical checkup information presented to the presentation target person, the output apparatus 23 may display, as a part of the recommended medical checkup information, an GUI including a display field 231 indicating the reason why the recommended medical checkup is recommended to the medical checkup target person.

For example, when the medical checkup selection unit 121 determines, as the additional information, the availability of the insurance for the recommended medical checkup as describe above, the medical checkup presentation unit 122 may control the information terminal 2 to present, to the presentation target person, the recommended medical checkup information indicating not only the item name of the recommended medical checkup but also the availability of the insurance for the recommended medical checkup. For example, as illustrated in FIG. 11, the output apparatus 23 may display, as a part of the recommended medical checkup information, an GUI including a display field 232 indicating the availability of the insurance for the recommended medical checkup.

For example, when the medical checkup selection unit 121 determines, as the additional information, the disease that is expected to be assessed by the recommended medical checkup as describe above, the medical checkup presentation unit 122 may control the information terminal 2 to present, to the presentation target person, the recommended medical checkup information indicating not only the item name of the recommended medical checkup but also the disease that is expected to be assessed by the recommended medical checkup. For example, as illustrated in FIG. 11, the output apparatus 23 may display, as a part of the recommended medical checkup information, an GUI including a display field 233 indicating the disease that is expected to be assessed by the recommended medical checkup.

For example, when the medical checkup selection unit 121 determines, as the additional information, the degree of the risk that the medical checkup target person catches the disease that is expected to be assessed by the recommended medical checkup as describe above, the medical checkup presentation unit 122 may control the information terminal 2 to present, to the presentation target person, the recommended medical checkup information indicating not only the item name of the recommended medical checkup but also the degree of the risk that the medical checkup target person catches the disease that is expected to be assessed by the recommended medical checkup. For example, as illustrated in FIG. 11, the output apparatus 23 may display, as a part of the recommended medical checkup information, an GUI including a display field 234 indicating the degree of the risk that the medical checkup target person catches the disease that is expected to be assessed by the recommended medical checkup.

For example, when the medical checkup selection unit 121 determines, as the additional information, the date on which the recommended medical checkup is recommended to be performed as describe above, the medical checkup presentation unit 122 may control the information terminal 2 to present, to the presentation target person, the recommended medical checkup information indicating not only the item name of the recommended medical checkup but also the date on which the recommended medical checkup is recommended to be performed. For example, as illustrated in FIG. 11, the output apparatus 23 may display, as a part of the recommended medical checkup information, an GUI including a display field 235 indicating the date on which the recommended medical checkup is recommended to be performed.

For example, when the medical checkup selection unit 121 determines, as the additional information, the degree of the recommendation of the recommended medical checkup as describe above, the medical checkup presentation unit 122 may control the information terminal 2 to present, to the presentation target person, the recommended medical checkup information indicating not only the item name of the recommended medical checkup but also the degree of the recommendation of the recommended medical checkup. For example, as illustrated in FIG. 11, the output apparatus 23 may display, as a part of the recommended medical checkup information, an GUI including a display field 236 indicating the degree of the recommendation of the recommended medical checkup.

When the medical checkup selection unit 121 selects the plurality of recommended medical checkups in a situation where the additional information is generated, the medical checkup presentation unit 122 may change the presentation content of the recommended medical checkup information based on the additional information. For example, the medical checkup presentation unit 122 may change an order of displaying the item names of the plurality of recommended medical checkups based on the additional information. For example, the medical checkup presentation unit 122 may change an order of displaying the additional information that are displayed in association with the item names of the plurality of recommended medical checkups based on the additional information. Specifically, for example, the medical checkup presentation unit 122 may display the item name of the recommended medical checkup (alternatively, the additional information related to the recommended medical checkup) at a higher level as the degree of the recommendation of the recommended medical checkup is higher (alternatively, the risk is higher or the recommended date is earlier). For example, the medical checkup presentation unit 122 may display one item name of the item names of the plurality of recommended medical checkups based on the additional information so that it is more highlighted than another item name of the item names of the plurality of recommended medical checkups. For example, the medical checkup presentation unit 122 may display the additional information related to one recommended medical checkup of the plurality of recommended medical checkups based on the additional information so that it is more highlighted than the additional information related to another recommended medical checkup of the plurality of recommended medical checkups. Specifically, for example, the medical checkup presentation unit 122 may display the item name of the recommended medical checkup (alternatively, the additional information related to the recommended medical checkup) with a more highlighted manner as the degree of the recommendation of the recommended medical checkup is higher (alternatively, the risk is higher or the recommended date is earlier).

When the medical checkup selection unit 121 selects the non-recommended medical checkup as described above, the medical checkup presentation unit 122 may control the information terminal 2 to present, to the presentation target person, the recommended medical checkup information indicating not only the item name of the recommended medical checkup but also an item name of the non-recommended medical checkup. For example, as illustrated in FIG. 11, the output apparatus 23 may display, as a part of the recommended medical checkup information, an GUI including a display field 237 indicating the item name of the non-recommended medical checkup. Furthermore, when the medical checkup selection unit 121 determines the reason why the non-recommended medical checkup is not recommended as described above, the medical checkup presentation unit 122 may control the information terminal 2 to present, to the presentation target person, the recommended medical checkup information indicating not only the item name of the recommended medical checkup but also the reason why the non-recommended medical checkup is not recommended. For example, as illustrated in FIG. 11, the output apparatus 23 may display, as a part of the recommended medical checkup information, an GUI including a display field 238 indicating the reason why the non-recommended medical checkup is not recommended.

The medical checkup presentation unit 122 may change the presentation content of the presented recommended medical checkup information depending on the presentation target person. For example, as described above, each of the medical checkup target person (typically, the patient) and the healthcare worker (typically, the doctor) may be the presentation target person. In this case, the medical checkup presentation unit 122 may control the information terminal 2 so that the presentation content of the recommended medical checkup information for the healthcare worker is different from the presentation content of the recommended medical checkup information for the patient. Namely, the medical checkup presentation unit 122 may control the information terminal 2 to present the recommended medical checkup information to the healthcare worker with a first presentation content and to present the recommended medical checkup information to the patient with a second presentation content.

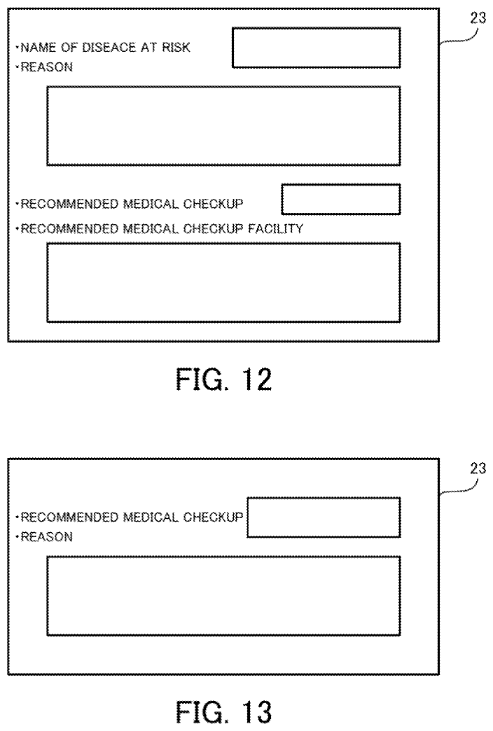

As one example, an information necessary for the healthcare worker is generally often more than an information necessary for the patient. Thus, the medical checkup presentation unit 122 may control the information terminal 2 so that an information amount of the recommended medical checkup information presented to the patient is smaller than an information amount of the recommended medical checkup information presented to the healthcare worker. The medical checkup presentation unit 122 may control the information terminal 2 so that a part of the recommended medical checkup information presented to the healthcare worker is presented to the patient and another part of the recommended medical checkup information presented to the healthcare worker is not presented to the patient. FIG. 12 illustrates one example of the recommended medical checkup information presented to the healthcare worker and FIG. 13 illustrates one example of the recommended medical checkup information presented to the patient. As illustrated in FIG. 12, the medical checkup presentation unit 122 may control the information terminal 2 to present, to the healthcare worker, the recommended medical checkup information that is not necessary for the patient but is necessary for the healthcare worker (for example, the recommended medical checkup information including the disease that is expected to be assessed by the recommended medical checkup (namely, the disease which the medical checkup target person has a risk to catch), a reason of the risk (namely, the reason why the recommended medical checkup is recommended) and the item name of the recommended medical checkup). Furthermore, in order to allow the healthcare worker to easily reserve a medical checkup facility in which the medical checkup target person takes the recommended medical checkup, the medical checkup presentation unit 122 may control the information terminal 2 to present, to the healthcare worker, a candidate of the medical checkup facility in which the recommended medical checkup can be performed. In this case, the medical checkup selection unit 121 may select the recommended medical checkup and may select the medical checkup facility in which the selected recommended medical checkup can be performed (especially, the medical checkup facility in which the selected recommended medical checkup is recommended to be performed) based on an information related to the medical checkup facility. On the other hand, as illustrated in FIG. 13, the medical checkup presentation unit 122 may control the information terminal 2 to present, to the patient, the recommended medical checkup information including the item name of the recommended medical checkup and the reason why the recommended medical checkup is recommended. In this case, the medical checkup presentation unit 122 may consider a possibility that the medical checkup target person recognizes the recommended medical checkup information by using the information terminal 2 at a place (for example, the home of the medical checkup target person and the like) that is different from the healthcare institution, and may control the information terminal 2 not to present, to the patient, an information that should not be legally presented to the patient in order to satisfy the legal constraint.

Alternatively, in an example illustrated in FIG. 13, a part of the recommended medical checkup information presented to the healthcare worker is same as the recommended medical checkup information presented to the patient. However, the medical checkup presentation unit 122 may present, to the healthcare worker, the recommended medical checkup information that is different from the recommended medical checkup information presented to the patient. For example, the medical checkup presentation unit 122 may present, to the healthcare worker, the recommended medical checkup information described by using a medical term that is understandable by the healthcare worker but that is difficult for the patient to easily understand. Similarly, the medical checkup presentation unit 122 may present, to the patient, the recommended medical checkup information that is different from the recommended medical checkup information presented to the healthcare worker. For example, the medical checkup presentation unit 122 may present, to the patient, the recommended medical checkup information described by using a simple work that is easily understandable without a medical expert knowledge.

When the recommended medical checkup information is presented to the healthcare worker, the healthcare worker may input a feedback related to an adequacy of the recommended medical checkup information to the medical checkup presentation server 1. The feedback from the healthcare worker may be used to select the recommended medical checkup by the medical checkup selection unit 121. For example, the feedback from the healthcare worker may be used to update the above described selection engine 123. For example, the feedback from the healthcare worker may be used to learn the parameter of the above described machine learning model 124.

(3) Technical Effect of Medical Checkup Presentation System SYS

As described above, the medical checkup presentation server 1 in the present example embodiment is capable of properly selecting the recommended medical checkup that is recommended to the medical checkup target person and presenting it to the presentation target person. Especially, the medical checkup presentation server 1 is capable of selecting the recommended medical checkup by using not only the medical chart data 1110 of the medical checkup target person but also the medical checkup history information 1123 related to the history of the medical checkups that have been taken in the past by the plurality of checked persons who includes the medical checkup target person or who are different from the medical checkup target person. Thus, the medical checkup presentation server 1 is capable of selecting the recommended medical checkup that is more suitable for the medical checkup target person using a past knowledge, compared to a case where the recommended medical checkup is selected without using the medical checkup history information 1123.

As one example, the medical checkup presentation server 1 may select, as the recommended medical checkup, the medical checkup which the medical checkup target person do not want to take or is reluctant to take generally, if needed. For example, the endoscopic test, which is preferably taken by the medical checkup target person for the early discovery of the large intestine cancer, is often avoided by the medical checkup target person because of an image that the endoscopic test is painful or frightening or an image that a cost of the endoscopic test is high. However, even when the endoscopic test is avoided by the medical checkup target person, the medical checkup presentation server 1 is capable of selecting the endoscopy test as the recommended medical checkup when it is determined that the endoscopic test is necessary from the past knowledge (namely, from the past medical checkup DB112). Thus, the medical checkup presentation server 1 is capable of greatly contributing to the early discovery or the prevention of the large intestine cancer. Incidentally, in order to properly select, as the recommended medical checkup, the endoscopy test for the early discovery or the prevention of the large intestine cancer, the medical checkup presentation server 1 may select the recommended medical checkup by using the medical chart data 1110 including the information related to the result of the medical interview (for example, an information on a lifestyle and the family history obtained by the medical interview) and an information related to the result of the test (for example, an information related to a result of a pathology reading).

As another example, even when a medical checkup is for determining whether or not the medical checkup target person is in a frail state a sign of which is difficult to detect (hereinafter, it is referred to as “a frailty test), the medical checkup presentation server 1 may select the frailty test as the recommended medical checkup when it is determined that the frailty test is necessary from the past knowledge (namely, from the past medical checkup DB112). Thus, the medical checkup presentation server 1 is capable of presenting the necessary medical checkup to an elderly medical checkup target person in advance so as to reduce an occurrence of a nursing care which is a major social issue in an aging society. Incidentally, in order to properly select the frailty test as the recommended medical checkup, the medical checkup presentation server 1 may select the recommended medical checkup by using the medical chart data 1110 including the information related to the life log (for example, an information related to an amount of exercise), the information related to the result of the medical interview (for example, the information on the lifestyle obtained by the medical interview) and the information related to the result of the medical examination (namely, the medical examination history information 1112).

Furthermore, the medical checkup presentation server 1 is capable of generating the additional information that may be presented to the presentation target person together with the item name of the recommended medical checkup, in addition to selectin the recommended medical checkup. Note that the information related to at least one of the reason why the recommended medical checkup is recommended, the availability of the insurance for the recommended medical checkup, the disease that is expected to be assessed by the recommend medical checkup, the degree of the risk that the medical checkup target person catches the disease that is expected to be assessed by the recommended medical checkup, the date on which the recommended medical checkup is recommended to be performed, the degree of the recommendation of the recommended medical checkup and the non-recommended medical checkup as described above is one example of the additional information, for example. In this case, the medical checkup presentation server 1 is capable of presenting, to the presentation target person, a beneficial information for the presentation target person to determine whether or not to take the recommended medical checkup.

Furthermore, the medical checkup presentation server 1 is capable of changing the presentation content of the recommended medical checkup depending on the presentation target person. Thus, the medical checkup presentation server 1 is capable of presenting the recommended medical checkup information with the presentation content that is suitable for the presentation target person.

(4) Supplementary Note

With respect to the example embodiments described above, the following Supplementary

Notes will be further disclosed.

### [Supplementary Note 1]

A medical checkup presentation apparatus including:

a selection unit that is configured to select, as a recommended medical checkup, a medical checkup that is recommended to a medical checkup target person based on medical chart data including an information related to a history of a medical examination for the medical checkup target person and past medical checkup data including an information related to a history of a medical checkup; and

a presentation unit that is configured to present, to a presentation target person, a recommended medical checkup information including an information related to an item name of the recommended medical checkup.

### [Supplementary Note 2]

The medical checkup presentation apparatus according to Supplementary Note 1, wherein

the presentation unit is configured to present, to a first presentation target person, the recommended medical checkup information with a first presentation content, and to present, to a second presentation target person that is different from the first presentation target person, the recommended medical checkup information with a second presentation content that is different from the first presentation content.

### [Supplementary Note 3]

The medical checkup presentation apparatus according to Supplementary Note 2, wherein

the first presentation target person is a healthcare worker,

the second presentation target person is the medical checkup target person.

### [Supplementary Note 4]

The medical checkup presentation apparatus according to Supplementary Note 2 or 3, wherein

an information amount of the recommended medical checkup information presented with the second presentation content is smaller than an information amount of the recommended medical checkup information presented with the first presentation content.

### [Supplementary Note 5]

The medical checkup presentation apparatus according to any one of Supplementary Notes 2 to 4, wherein

the recommended medical checkup information presented with the second presentation content is a part of the recommended medical checkup information presented with the first presentation content.

### [Supplementary Note 6]

The medical checkup presentation apparatus according to any one of Supplementary Notes 1 to 5, wherein

the presentation unit is configured to present the recommended medical checkup information by using a presentation device located on a traffic line of the medical checkup target person in a healthcare institution.

### [Supplementary Note 7]

The medical checkup presentation apparatus according to any one of Supplementary Notes 1 to 6, wherein

the presentation unit is configured to present the recommended medical checkup information by using a presentation device located in at least one of a reception, a waiting room and a medical consultation room in a healthcare institution.

### [Supplementary Note 8]

The medical checkup presentation apparatus according to any one of Supplementary Notes 1 to 7, wherein

the presentation unit is configured to present the recommended medical checkup information by using a presentation device of a healthcare institution, a presentation device lent to the medical checkup target person by the healthcare institution and a presentation device of the medical checkup target person.

### [Supplementary Note 9]

The medical checkup presentation apparatus according to any one of Supplementary Notes 1 to 8, wherein

the recommended medical checkup information further includes at least one of an information related to a reason why the recommended medical checkup is recommended, an information related to an availability of an insurance for the recommended medical checkup, an information related to a disease that is expected to be assessed by the recommended medical checkup, an information related to a degree of a risk that the medical checkup target person catches the disease that is expected to be assessed by the recommended medical checkup, an information related to a date on which the recommended medical checkup is recommended to be performed and an information related to a degree of a recommendation of the recommended medical checkup.

### [Supplementary Note 10]

The medical checkup presentation apparatus according to any one of Supplementary Notes 1 to 9, wherein

the medical chart data further includes at least one of an information related to a result of a health check taken by the medical checkup target person, an information related to a result of a medical interview for the medical checkup target person, an information related to a life log of the medical checkup target person, an information related to a personal information of the medical checkup target person and an information related to a medical checkup that has been taken by the medical checkup target person in the past.

### [Supplementary Note 11]

The medical checkup presentation apparatus according to any one of Supplementary Notes 1 to 10, wherein

the past medical checkup data further includes at least one of an information related to a history of a medical examination of a checked person who has taken the medical checkup in the past, an information related to a result of a health check of the checked person, an information related to a result of a medical interview for the checked person, an information related to a life log of the checked person, an information related to a personal information of the checked person and an information related to a medical checkup that is recommended by a healthcare institute.

### [Supplementary Note 12]

A medical checkup presentation method including:

selecting, as a recommended medical checkup, a medical checkup that is recommended to a medical checkup target person based on medical chart data including an information related to a history of a medical examination for the medical checkup target person and past medical checkup data including an information related to a history of a medical checkup; and

presenting, to a presentation target person, a recommended medical checkup information including an information related to an item name of the recommended medical checkup.

### [Supplementary Note 13]

A recording medium on which a computer program that allows a computer to execute:

a selection step at which a medical checkup that is recommended to a medical checkup target person is selected as a recommended medical checkup based on medical chart data including an information related to a history of a medical examination for the medical checkup target person and past medical checkup data including an information related to a history of a medical checkup; and

a presentation step at which a recommended medical checkup information including an information related to an item name of the recommended medical checkup is presented to a presentation target person

is recorded.

### [Supplementary Note 13]

A computer program that allows a computer to execute:

a selection step at which a medical checkup that is recommended to a medical checkup target person is selected as a recommended medical checkup based on medical chart data including an information related to a history of a medical examination for the medical checkup target person and past medical checkup data including an information related to a history of a medical checkup; and

a presentation step at which a recommended medical checkup information including an information related to an item name of the recommended medical checkup is presented to a presentation target person.

This disclosure is not limited to the above described example embodiment. This disclosure is allowed to be changed, if desired, without departing from the essence or spirit of the invention which can be read from the claims and the entire specification, and a medical checkup presentation apparatus, a medical checkup presentation method and a recording medium, which involve such changes, are also intended to be within the technical scope of this disclosure.

This application is based upon and claims the benefit of priority from Japanese Patent Application No. 2020-097582, filed on Jun. 4, 2020, and incorporates all of its disclosure herein. Moreover, this application incorporates all of the publications of application and articles.

### DESCRIPTION OF REFERENCE CODES

SYS medical checkup presentation system
1 medical checkup presentation server
11 storage apparatus
111 medical chart DB
1110 medical chart data
1111 patient basic information
1112 medical examination history information
112 past medical checkup DB
1120 past medical checkup data
1121 checked person basic data
1122 medical examination history information
1123 medical checkup history information
12 arithmetic apparatus
121 medical checkup selection unit
122 medical checkup presentation unit
2 information terminal
22 arithmetic apparatus
23 output apparatus

What is claimed is:

1. A medical checkup presentation apparatus comprising:
at least one memory configured to store instructions; and
at least one processor configured to execute the instructions to:
select, as a recommended medical checkup, a medical checkup that is recommended to a medical checkup target person based on medical chart data including an information related to a history of a medical examination for the medical checkup target person and past medical checkup data including an information related to a history of a medical checkup; and
present, to a presentation target person, a recommended medical checkup information including an information related to an item name of the recommended medical checkup.

2. The medical checkup presentation apparatus according to claim 1, wherein
the at least one processor is configured to execute the instructions to present, to a first presentation target person, the recommended medical checkup information with a first presentation content, and to present, to a second presentation target person that is different from the first presentation target person, the recommended medical checkup information with a second presentation content that is different from the first presentation content.

3. The medical checkup presentation apparatus according to claim 2, wherein
the first presentation target person is a healthcare worker,
the second presentation target person is the medical checkup target person.

4. The medical checkup presentation apparatus according to claim 2, wherein
an information amount of the recommended medical checkup information presented with the second presentation content is smaller than an information amount of the recommended medical checkup information presented with the first presentation content.

5. The medical checkup presentation apparatus according to claim 2, wherein
the recommended medical checkup information presented with the second presentation content is a part of the recommended medical checkup information presented with the first presentation content.

6. The medical checkup presentation apparatus according to claim 1, wherein
the at least one processor is configured to execute the instructions to present the recommended medical checkup information by using a presentation device located on a traffic line of the medical checkup target person in a healthcare institution.

7. The medical checkup presentation apparatus according to claim 1, wherein
the at least one processor is configured to execute the instructions to present the recommended medical checkup information by using a presentation device located in at least one of a reception, a waiting room and a medical consultation room in a healthcare institution.

8. The medical checkup presentation apparatus according to claim 1 wherein
the at least one processor is configured to execute the instructions to present the recommended medical checkup information by using a presentation device of a healthcare institution, a presentation device lent to the medical checkup target person by the healthcare institution and a presentation device of the medical checkup target person.

9. The medical checkup presentation apparatus according to claim 1, wherein
the recommended medical checkup information further includes at least one of an information related to a reason why the recommended medical checkup is recommended, an information related to an availability of an insurance for the recommended medical checkup, an information related to a disease that is expected to be assessed by the recommended medical checkup, an information related to a degree of a risk that the medical checkup target person catches the disease that is expected to be assessed by the recommended medical checkup, an information related to a day on which the recommended medical checkup is recommended to be taken and an information related to a degree of a recommendation of the recommended medical checkup.

10. The medical checkup presentation apparatus according to claim 1, wherein
the medical chart data further includes at least one of an information related to a result of a health check taken by the medical checkup target person, an information related to a result of a medical interview for the medical checkup target person, an information related to a life log of the medical checkup target person, an information related to a personal information of the medical checkup target person and an information related to a medical checkup that has been taken by the medical checkup target person in the past.

11. The medical checkup presentation apparatus according to claim 1, wherein
the past medical checkup data further includes at least one of an information related to a history of a medical examination of a checked person who has taken the medical checkup in the past, an information related to a result of a health check of the checked person, an information related to a result of a medical interview for the checked person, an information related to a life log of the checked person, an information related to a personal information of the checked person and an information related to a medical checkup that is recommended by a healthcare institute.

12. A medical checkup presentation method comprising:
selecting, as a recommended medical checkup, a medical checkup that is recommended to a medical checkup target person based on medical chart data including an information related to a history of a medical examination for the medical checkup target person and past medical checkup data including an information related to a history of a medical checkup; and
presenting, to a presentation target person, a recommended medical checkup information including an information related to an item name of the recommended medical checkup.

13. A non-transitory recording medium on which a computer program that allows a computer to execute a medical checkup presentation method is recorded,
the medical checkup presentation method including:
selecting a medical checkup that is recommended to a medical checkup target person as a recommended medical checkup based on medical chart data including an information related to a history of a medical examination for the medical checkup target person and past medical checkup data including an information related to a history of a medical checkup; and
presenting a recommended medical checkup information including an information related to an item name of the recommended medical checkup to a presentation target person.
